(19) United States
(12) Patent Application Publication
(10) Pub. No.: US 2026/0183361 A1
(43) Pub. Date: Jul. 2, 2026

(54) CONJUGATION OF SACCHARIDE ANTIGENS USING ACETOXYBOROHYDRIDES

(71) Applicant: MERCK SHARP & DOHME LLC, Rahway, NJ (US)

(72) Inventors: Katherine M. PHILLIPS, Audubon, PA (US); Adriana N. SANTIAGO-MIRANDA, Conshohocken, PA (US); Chengli ZONG, Haikou (CN); Jacob Henry WALDMAN, Metuchen, NJ (US); Patrick MCHUGH, Pipersville, PA (US); John LIMANTO, Jersey City, NJ (US); Jian HE, Blue Bell, PA (US)

(73) Assignee: MERCK SHARP & DOHME LLC, Rahway, NJ (US)

(21) Appl. No.: 19/123,846

(22) PCT Filed: Nov. 2, 2023
(86) PCT No.: PCT/US2023/078432

§ 371 Date: Apr. 24, 2025

Related U.S. Application Data
(60) Provisional application No. 63/423,295, filed on Nov. 7, 2022.

(51) Int. Cl. A61K 38/00; A61K 39/00; A61K 39/09; A61K 47/64

(52) U.S. Cl. CPC A61K 38/00; A61K 39/092; A61K 47/64; A61K 47/6415; A61K 2039/6037

(57) ABSTRACT

The present disclosure relates generally to improved methods of preparing glycoconjugates. The methods comprise using a reducing mixture containing acetoxyborohydrides prepared in-situ for conjugating a saccharide to a carrier protein.

### FIELD OF THE INVENTION

The invention relates to methods of preparing glycoconjugates by conjugating a polysaccharide to a carrier protein using in-situ prepared acetoxyborohydrides mediated reductive amination.

### BACKGROUND OF THE INVENTION

Bacterial capsular polysaccharides are long polymers composed of many repeating units of simple sugars, which help protect the bacteria from phagocytosis. Antibodies against capsular polysaccharides of many pathogenic bacteria can increase phagocytosis of bacteria, thereby stimulating an immune response. However, while vaccines composed of purified polysaccharides are partially immunogenic in adults, they fail to induce an antibody response in infants and children. This problem is overcome by chemically conjugating the polysaccharides to a carrier protein, thereby making the polysaccharide more immunogenic. Coupling converts the polysaccharide, a T-independent antigen, to a protein, a T-dependent antigen, which allows for isotype switching, affinity maturation, and formation of memory B cells.

The two most widely used methods of conjugating bacterial polysaccharides to carrier proteins involve amidation and reductive amination. In the method involving amidation, the reducing end of the polysaccharide is first oxidized to the corresponding aldonic acid. This step is followed by reaction of the acid group of the aldonic acid with the primary amine in the lysine side chain of the carrier protein. In the method involving reductive amination, the reducing end of the polysaccharide is first oxidized to the corresponding aldehyde. This reaction is followed by the formation of a Schiff base between the aldehyde group and the primary amine in the lysine side chain of the carrier protein (or the N-terminal amine) to generate an imine, and reduction of the imine with a hydride source. Cyanoborohydride has been used as the hydride source for the conjugation of bacterial polysaccharides, e.g., Streptococcus pneumoniae saccharides (WO 1987/006838). However, use of cyanoborohydride has disadvantages such as slow reaction time and contamination of the amine produced with cyanide (Ahmed F. et al. J. Org. Chem., 1996, 61:3849-3862).

Triacetoxyborohydride has been used as an alternative to cyanoborohydride for the reduction of aldehydes and ketones (Ahmed F. et al. J Org. Chem., 1996, 61:3849-3862), and carbohydrates (Dalpathado et al. Anal. Bioanal. Chem. (2005) 281:130-1137). Triacetoxyborohydride has been used also to reduce the imine formed during conjugation of capsular polysaccharide to a protein carrier (EP 2683408). However, there is a need for improvement in the efficiency of triacetoxyborohydride mediated reductive amination.

### SUMMARY OF THE INVENTION

Accordingly, the present invention provides an improved method of conjugating a saccharide antigen to a carrier protein using acetoxyborohydrides. The improved method comprises using a reducing mixture comprising freshly or in situ prepared acetoxyborohydrides for reductive amination of the Schiff base formed between the saccharide and the carrier protein during conjugation.

In one aspect, the disclosure provides a method for conjugating an antigen to a carrier protein. The method comprises the steps of (a) activating the antigen to form an activated antigen; (b) reacting the activated antigen with a carrier protein to obtain an intermediate in which the activated antigen and the carrier protein are linked by an imine group; and (c) reducing the imine group by a process that includes (i) mixing acetic acid with a borohydride solution to prepare a reducing mixture comprising acetoxyborohydrides and (ii) treating the intermediate with the reducing mixture. At least about 20% of the acetoxyborohydrides in the reducing mixture used to treat the intermediate is diacetoxyborohydride (DAB). The antigen is a saccharide. The reducing mixture is alternatively referred to herein as in-situ acetoxyborohydrides reducing mixture.

In some embodiments, the remainder of the acetoxyborohydride in the reducing mixture is either tricetoxyborohydride (TAB) or a mixture of TAB and monoacetoxyborohydride (MAB).

In some embodiments, the saccharide is a bacterial capsular polysaccharide.

In some embodiments, the reducing mixture is held for a period of time (i.e., not used for some time following preparation of said reducing mixture), e.g., at least about 30 minutes, about 1 to about 8 hours, or about 2 to about 6 hours before using it for treating the intermediate.

In some embodiments, the borohydride used is sodium borohydride or potassium borohydride. In some embodiments, the borohydride solution is prepared by dissolving the borohydride in dimethylsulphoxide (DMSO).

In some embodiments, step (c)(i) is carried out at a temperature of about 20° C. to about 35° C., about 20° C. to about 30° C., or about 20° C. to about 25° C.

In some embodiments, unreacted carbonyl groups (e.g., residual aldehyde groups) are reduced by the reducing mixture. In some embodiments, the method further comprises reducing unreacted carbonyl groups using a borohydride (e.g., sodium borohydride or potassium borohydride). In some embodiments, the reducing mixture includes at least about 30% diacetoxyborohydride.

In some embodiments, the bacterial capsular saccharide conjugated according to the above method originates from Streptococcus pneumoniae, Haemophilus influenzae, Neisseria meningitidis, Staphylococcus aureus, Enterococcus faecium, Enterococcus faecalis, Salmonella vi, or Staphylococcus epidermidis.

In some embodiments, the carrier protein may be tetanus toxoid (TT), fragment C of tetanus toxoid, diphtheria toxoid (DT), CRM197, Pneumolvsin (Ply). protein D, PhtD (Pneumococcal histidine triad protein D), PhtDE, or N19. In some embodiments, the carrier protein is CRM197, and the saccharide is conjugated to lysine residues of the CRM197 to yield a molar ratio of conjugated CRM197 lysine residues to total CRM197 amine residues of between about 0.5:10 to about 5:10.

In some embodiments, the conjugated antigen prepared according to the methods of the invention has a molecular weight of between about 50 kDa and about 20,000 kDa. In some embodiments, the conjugated antigen has less than about 45% free bacterial capsular polysaccharide compared to the total amount of the bacterial capsular polysaccharide.

In another aspect, the disclosure provides an immunogenic composition that includes a conjugated antigen or a mixture of conjugated antigens, one or more of which are prepared according to the method of conjugation described herein, mixed with a pharmaceutically acceptable excipient.

### BRIEF DESCRIPTION OF THE DRAWINGS

FIG. 1 is a graph showing variation in the concentration of the acetoxyborohydrides, SMAB (sodium monoacetoxyborohydride), SDAB (sodium diacetoxyborohydride), and STAB (sodium triacetoxyborohydride) in the in-situ acetoxyborohydrides reducing mixture with time. FIG. 1 also shows the relative amounts of SDAB and STAB present in commercially available STAB.

FIG. 2 shows variation in the size of the conjugate formed as a function of time the in-situ acetoxyborohydrides reducing mixture is held before using it for reducing the imine group in a conjugation reaction.

FIG. 3 shows the effect of temperature under which the in-situ acetoxyborohydrides reducing mixture is prepared on conjugation efficiency. The conjugation efficiency is measured by the size of the conjugate produced (n=3).

FIG. 4 (FIG. 4A, FIG. 4B, and FIG. 4C) are graphs showing functional antibody titers (OPA Titer) in mice immunized with pneumococcal serotypes 9N, 22F, and 35B. In the conjugation of these serotypes the in-situ acetoxyborohydrides reducing mixture was either used (Arm 2) or not used (Arm 1). In Arm 1, for serotypes 9N and 22F, cyanoborohydride was used as the reducing agent, and for serotype 35B, no reducing agent was used.

### DETAILED DESCRIPTION OF THE INVENTION

The present disclosure may be understood more readily by reference to the following detailed description of the various embodiments of the disclosure and the examples included herein. Unless defined otherwise, all technical and scientific terms used herein have the same meaning as commonly understood by one of ordinary skill in the art to which the disclosure pertains. In describing the embodiments and in the claims, certain terminology will be used in accordance with the definitions set out below.

As used herein, the singular forms “a”, “an”, and “the” include plural references unless indicated otherwise. Thus, for example, references to “the method” includes one or more methods, and/or steps of the type described herein and/or which will become apparent to one of ordinary skill in the art upon reading this disclosure.

As used herein, the term “about” means within a statistically meaningful range of a value, such as a stated concentration range, time frame, molecular weight, temperature, or pH. Such a range can be within an order of magnitude, typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study and can be readily appreciated by one of ordinary skill in the art. Whenever a range is recited within this application, each of the endpoints of the range and every value within the range is also contemplated as an embodiment of the disclosure.

It is noted that in this disclosure, terms such as “comprises,” “comprised,” “comprising,” “contains,” “containing” and the like mean “includes,” “included.” “including” and the like. Such terms refer to the inclusion of particular ingredients or set of ingredients without excluding any other ingredients.

Terms such as “consisting essentially of” and “consists essentially of” allow for the inclusion of additional ingredients or steps that do not detract from the novel or basic characteristics of the disclosure, i.e., they exclude additional unrecited ingredients or steps that detract from the novel or basic characteristics of the disclosure. The terms “consists of” and “consisting of” are close ended terms. Accordingly, these terms refer to the inclusion of a particular ingredient or set of ingredients and the exclusion of all other ingredients.

As used herein, the term “saccharide” is used to refer to a polysaccharide, an oligosaccharide, or a monosaccharide.

The term “conjugates” or “glycoconjugates” as used herein refers to a saccharide covalently conjugated to a carrier protein. Glycoconjugates of the disclosure and immunogenic compositions comprising them may contain some amount of free saccharide.

The term “free saccharide” as used herein means a saccharide that is not covalently conjugated to the carrier protein but is nevertheless present in the glycoconjugate composition. The free saccharide may be non-covalently associated with (i.e., non-covalently bound to, adsorbed to, or entrapped in or with) the conjugated saccharide-carrier protein glycoconjugate. The terms “free polysaccharide” and “free capsular polysaccharide” are used herein to convey the same meaning with respect to glycoconjugates wherein the saccharide is a polysaccharide or a capsular polysaccharide, respectively.

As used herein, “freshly or in situ prepared acetoxyborohydrides” refers to a mixture of acetoxyborohydrides comprising di-, tri-, and optionally, mono-acetoxyborohydrides, prepared by mixing a borohydride (e.g., sodium borohydride) and acetic acid less than 25 hours before use in a conjugation reaction. For example, the acetoxyborohydrides may be prepared 10 hours, 8 hours, 6 hours, 4 hours, 2 hours, 1 hour, or 30 minutes before use.

As used herein, “to conjugate,” “conjugated” and “conjugating” refer to a process whereby a saccharide, for example a bacterial capsular polysaccharide, is covalently attached to a carrier molecule or carrier protein. The conjugation can be performed according to the methods described below or by other processes known in the art. Conjugation enhances the immunogenicity of the bacterial capsular polysaccharide.

The term “subject” refers to a mammal, including a human, or to a bird, fish, reptile, amphibian or any other animal. The term “subject” also includes household pets or research animals. Non-limiting examples of household pets and research animals include dogs, cats, pigs, rabbits, rats, mice, gerbils, hamsters, guinea pigs, ferrets, monkeys, birds, snakes, lizards, fish, turtles, and frogs. The term “subject” also includes livestock animals. Non-limiting examples of livestock animals include alpaca, bison, camel, cattle, deer, pigs, horses, llamas, mules, donkeys, sheep, goats, rabbits, reindeer, yak, chickens, geese, and turkeys.

### Glycoconjugates

The present disclosure relates to methods of conjugating a saccharide to a carrier protein, i.e., preparing a glycoconjugate, in particular, by using a reducing mixture prepared freshly or in situ comprising di-, tri-, and optionally, mono-acetoxyborohydrides, to reduce the imine group formed during the conjugation process.

In the glycoconjugates of the disclosure, the saccharide may be a monosaccharide, an oligosaccharide, or a polysaccharide, and the carrier protein may be any suitable carrier protein as further described herein or known to those of skill in the art. In some embodiments, the saccharide is a polysaccharide, in particular, a bacterial capsular polysaccharide, such as one from Streptococcus pneumoniae (S. pneumoniae; serotype 35B). In some embodiments, the carrier protein is CRM197 (cross-reactive material 197 from Corynebacterium diphtheriae C7).

Capsular polysaccharides can be prepared by standard techniques known to those skilled in the art and from a variety of serotypes, for example, serotypes 1, 3, 4, 5, 6A, 6B, 7F, 8, 9V, 10A, 11A, 12F, 14, 15B, 18C, 19A, 19F, 22F, 23F and 33F of S. pneumoniae. Conjugates can be prepared by separate processes and formulated into a single dosage formulation. For example, each pneumococcal polysaccharide serotype may be grown separately, and the individual polysaccharides may then be purified through steps including one or more of centrifugation, precipitation, ultra-filtration, and column chromatography. The purified polysaccharides may be chemically activated to make the saccharides capable of reacting with the carrier protein. Once activated, each capsular polysaccharide may be separately conjugated to a carrier protein to form a glycoconjugate. Each capsular polysaccharide in a formulation may be conjugated to the same carrier protein. Alternatively, more than one carrier protein may be used for conjugation of the polysaccharides. The chemical activation of the polysaccharides may be achieved by conventional means. See, for example, U.S. Pat. Nos. 4,902,506, 7,709,001, and 7,955,605.

In one embodiment, the glycoconjugate of the disclosure has a molecular weight of between about 50 kDa and about 20,000 kDa. In another embodiment, the glycoconjugate has a molecular weight of between about 200 kDa and about 10,000 kDa. In another embodiment, the gly coconjugate has a molecular weight of between about 500 kDa and about 5.000 kDa. In one embodiment, the glycoconjugate has a molecular weight of between about 1,000 kDa and about 3,000 kDa. In other embodiments the glycoconjugate has a molecular weight of between about 600 kDa and about 2800 kDa; between about 700 kDa and about 2700 kDa; between about 1000 kDa and about 2000 kDa; between about 1800 kDa and about 2500 kDa; between about 1100 kDa and about 2200 kDa; between about 1900 kDa and about 2700 kDa; between about 1200 kDa and about 2400 kDa; between about 1700 kDa and about 2600 kDa; between about 1300 kDa and about 2600 kDa; between about 1600 kDa and about 3000 kDa. Any whole number integer within any of the above ranges is contemplated as an embodiment of the disclosure.

In one embodiment, the polysaccharide is a capsular polysaccharide derived from Neisseria meningitidis (N. meningitidis). In some such embodiments, the capsular polysaccharide is selected from the group consisting of serotypes A, B, C, W135, X and Y capsular polysaccharides of N. meningitidis.

In one embodiment, the polysaccharide is a capsular polysaccharide derived from S. pneumoniae. For example, the capsular polysaccharide is from the S. pneumoniae serotype 1, 2, 3, 4, 5, 6A, 6B, 7F, 8, 9N, 9V, 10A, 11A, 12F, 14, 15A, 15B, DeOAc15B, 16F, 17F, 18C, 19A, 19F, 20, 22F, 23A, 23F, 23B, 24F, 31, 24F, 31, 33F, or 35B.

As used herein, DeOAc15B (De-O-acetylated serotype) refers to a pneumococcal polysaccharide that is substantially equivalent to serotype 15C pneumococcal polysaccharide and has a substantially identical NMR spectrum (data not shown). As used herein, de-O-acetylated serotype 15B pneumococcal polysaccharide and serotype 15C pneumococcal polysaccharide may each have an O-Acetyl content per repeating unit in the range of 0-5%, or in the range of 04%, or in the range of 0-3%, or in the range of 0-2%, or in the range of 0-1%, or in the range of 0-0.5%, or in the range of 0-0.1%, or no O-acetyl content. In a report by Spencer B. L., et al., pneumococcal polysaccharide 15C may be slightly 0-acetylated (Spencer, B. L. et al., Clin. Vac. Immuno. (2017) 24(8): 1-13). Thus, in any of the embodiments of the method described herein, de-O-acetylated serotype 15B (DeOAc15B) can be used in place of serotype 15C. Processes for de-O-acetylation are known in the art, for example as described in Rajam et al., Clinical and Vaccine Immunology, 2007, 14(9): 1223-1227.

In some embodiments, the glycoconjugate of the disclosure comprises a bacterial capsular polysaccharide, wherein the capsular polysaccharide has a molecular weight of between 10 kDa and 2,000 kDa or between 50 kDa and 1,000 kDa.

Glycoconjugates comprising a capsular polysaccharide covalently conjugated to a carrier protein produced by a method of the invention may have one or more of the following features: the molecular weight of the polysaccharide is between 50 kDa and 1,000 kDa; the glycoconjugate molecular weight is between 1,000 kDa to 5,000 KDa; and the conjugate comprises less than about 45% free polysaccharide relative to total polysaccharide. In some embodiments, the polysaccharide has a molecular weight of between 10 kDa and 2,000 kDa. In some embodiments, the glycoconjugate has a molecular weight of between 50 kDa and 20,000 kDa. In other embodiments, the glycoconjugate has a molecular weight of between 200 kDa and 10.000 kDa. In other embodiments, the conjugate comprises less than about 30%, 20%, 15%, 10%, or 5% free polysaccharide relative to total polysaccharide. The amount of free polysaccharide can be measured as a function of time, for example after 10, 20, 30, 40, 50, 60, 70, 80, 90, or 120 days, or even longer, after the conjugate was prepared.

The number of lysine residues in the carrier protein conjugated to the saccharide can be characterized as a range of conjugated lysine residues, which may be expressed as a molar ratio. The carrier protein may be CRM197, which contains 39 lysine amines. All these amines and the N-terminal amine (i.e., a total of 40 amine residues) can serve as sites of conjugation. However, not all these amine residues are equally reactive. In some embodiments, in a glycoconjugate made with CRM197, only 4 to 15 lysine residues are covalently linked to the saccharide, resulting in a molar ratio of conjugated lysine residues to total CRM197 amine residues of between about 1:10 to about 4:10. In another embodiment, 2 to 20 lysine residues of CRM197 are covalently linked to the saccharide, resulting in a molar ratio of conjugated lysine residues to CRM197 amine residues of between about 0.5:10 to about 5:10.

The saccharide:carrier protein ratio (w/w) also may vary. In one embodiment, the saccharide:carrier protein ratio (w/w) is between 0.2 and 4. In another embodiment, the saccharide:carrier protein ratio (w/w) is between 1.1 and 1.7. In some embodiments, the saccharide is a bacterial capsular polysaccharide, and the saccharide:carrier protein ratio (w/w) is between 0.2 and 4. In other embodiments, the saccharide is a bacterial capsular polysaccharide, and the saccharide:carrier protein ratio (w/w) is between 1.1 and 1.7. In some such embodiments, the carrier protein is CRM197.

The frequency of attachment of the saccharide chain to lysine residues on the carrier protein also may vary. For example, in one embodiment, there is at least one covalent linkage between the carrier protein and the polysaccharide for every 100 saccharide repeat units of the polysaccharide. In one embodiment, there is at least one covalent linkage between the carrier protein and the polysaccharide for every 50 saccharide repeat units of the polysaccharide. In one embodiment, there is at least one covalent linkage between the carrier protein and the polysaccharide for every 25 saccharide repeat units of the polysaccharide. In another embodiment, the covalent linkage between the carrier protein and the polysaccharide occurs at least once in every 4 saccharide repeat units of the polysaccharide. In another embodiment, the covalent linkage between the carrier protein and the polysaccharide occurs at least once in every 10 saccharide repeat units of the polysaccharide. In a further embodiment, the covalent linkage between the carrier protein and the polysaccharide occurs at least once in every 15 saccharide repeat units of the polysaccharide. In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide.

In some embodiments, the carrier protein is CRM197 and the covalent linkage between the CRM197 and the polysaccharide occurs at least once in every 4, 10, 15, or 25 saccharide repeat units of the polysaccharide. In some such embodiments, the polysaccharide is a bacterial capsular polysaccharide, for example a capsular polysaccharide derived from S. pneumoniae or N. meningitidis bacteria.

In one embodiment, the glycoconjugate comprises less than about 45% free saccharide compared to the total amount of saccharide. In another embodiment, the glycoconjugate comprises less than about 30% free saccharide compared to the total amount of saccharide. In another embodiment, the glycoconjugate comprises less than about 20% free saccharide compared to the total amount of saccharide. In a further embodiment, the glycoconjugate comprises less than about 10% free saccharide compared to the total amount of saccharide. In another embodiment, the glycoconjugate comprises less than about 5% free saccharide compared to the total amount of saccharide.

In some embodiments, the glycoconjugate comprises less than about 30 mole %, less than about 25 mole %, less than about 20 mole %, less than about 15 mole %, or less than about 10 mole % of carrier protein residues compared to the total amount of glycoconjugate.

### Carrier Protein

The term “protein carrier” or “carrier protein” or “carrier” refers to any protein molecule that may be conjugated to an antigen (such as a capsular polysaccharide) against which an immune response is desired.

Conjugation to a carrier can enhance the immunogenicity of the antigen. Protein carriers for the antigens can be toxins, toxoids, or any mutant cross-reactive material (CRM) of the toxin from tetanus, diphtheria, pertussis. Pseudomonas, E. coli, Staphylococcus and Streptococcus. In one embodiment, the carrier is diphtheria toxoid CRM.sub.197, derived from C. diphtheriae strain C7 (6197), which produces CRM.sub.197 protein. This strain has ATCC Accession No. 53281. A method for producing CRM.sub.197 is described in U.S. Pat. No. 5,614,382, which is incorporated herein by reference in its entirety. Alternatively, a fragment or epitope of the protein carrier or other immunogenic protein can be used. For example, a haptenic antigen can be coupled to a T-cell epitope of a bacterial toxin, toxoid, or CRM. Other suitable carrier proteins include inactivated bacterial toxins such as cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumolysin, pneumococcal surface protein A (PspA), pneumococcal adhesion protein (PsaA), or Haemophilus influenzae protein D can also be used. Other proteins, such as ovalbumin, keyhole limpet hemocyanin (KLH), bovine serum albumin (BSA) or purified protein derivative of tuberculin (PPD) also can be used as carrier proteins.

### Characterization of Polysaccharide-Protein Conjugates

A glycoconjugate may be characterized based on its molecular weight, which can be either number averaged molecular weight (Mn) or weight averaged molecular weight (Mw). A glycoconjugate is a polymer that exists as a distribution of chain lengths and molecular weights. As such, it is not possible to have an exact molecular weight of the glycoconjugate. Instead averages of different parameters are used to indicate the molecular weight of a glycoconjugate. Mn and Mw are such two averages. Mn is the statistical average molecular weight of all the polymer chains in the sample. It is obtained by dividing the total weight of polymer by the total number of molecules. On the other hand, Mw, takes into consideration the molecular weight of a polymer chain. It is based on the fact that a molecule with greater mass contains more of the total mass of the polymer sample than the smaller molecules. Thus, Mw is a weighted average, and reflects the weight, on average, of a molecule in the polymer sample. Different methods are used to determine Mn and Mw. For a glycoconjugate, Mn can be measured using size exclusion chromatography and Mw can be measured using static light scattering. Both Mn and Mw can be measured simultaneously using size-exclusion chromatography (SEC) followed by continuous monitoring of differential refractive index (RI), ultraviolet light absorbance (UV), and multi-angle laser light scattering (MALS). Pollock J F et al., Bioconjug Chem. 2012 Sep. 19; 23(9): 1794-1801

Glycoconjugates may also be characterized by the number of lysine residues in the carrier protein that become conjugated to the saccharide. This number may be represented as a range of conjugated lysine residues (degree of conjugation). The evidence for lysine modification of the carrier protein, due to covalent linkages to the polysaccharides, can be obtained by amino acid analysis using routine methods known to those of ordinary skill in the art. Conjugation results in a reduction in the number of lysine residues recovered compared to the carrier protein starting material used to generate the conjugate materials. For example, the degree of conjugation may be as low as 2 or as high as 15.

Another method of characterizing glycoconjugates is by using the ratio (weight/weight) of saccharide to the carrier protein (Ps:Pr). For example, the Ps:Pr may be as low as 0.5 or as high as 3.0. The glycoconjugates may contain free saccharide that is not covalently conjugated to the carrier protein but is nevertheless present in the glycoconjugate composition. The free saccharide may be non-covalently associated with (i.e., non-covalently bound to, adsorbed to, or entrapped in or with) the glycoconjugate. For example, the glycoconjugate may comprise less than about 50%, 45%, 40%, 35%, 30%, 25%, 20% or 15% of free polysaccharide compared to the total amount of polysaccharide.

### Conjugation

To make a conjugate with a carrier protein, a polysaccharide must be activated (i.e., chemically modified) before it can be chemically linked to the carrier, such as a protein. Prior to the activation step, saccharides may be hydrolyzed or mechanically sized by pressure homogenization to achieve appropriate molecular weights (e.g., 50 kDa to 500 kDa) for activation and subsequent conjugation. Partial oxidation of carbohydrates in polysaccharides has been effectively utilized to generate aldehyde groups which are then coupled to amine groups, such as the lysine residues of carrier proteins, to generate immunogenic conjugates. It is important that the method used to conjugate a polysaccharide to a carrier protein result in a stable covalent linkage, and that the reaction conditions be mild enough to maintain the structural integrity of the individual components. Methods commonly used to activate and couple polysaccharides to carrier proteins include reductive amination chemistry (RAC), cyanylation, and use of carbodiimide. Reductive amination typically involves the use of sodium or potassium periodate or periodic acid to selectively oxidize vicinal —OH groups into active aldehyde groups. Cyanylation is used to randomly convert —OH groups into active —CN groups. Carbodiimide is used to activate carboxyl groups by replacing —OH groups with carbodiimide.

Reductive amination chemistry (RAC) is one of the most commonly used methods to couple polysaccharides to proteins since the reaction between the resulting carbonyl group of the polysaccharide and the amino group of the carrier protein can form corresponding Schiff base, which is generally selectively reduced in the presence of sodium cyanoborohydride to a stable saturated carbon-nitrogen bond. Furthermore, reductive amination can be carried out in aqueous solution under conditions mild enough to preserve the structural integrity of the saccharide and protein components. Following conjugation, unreacted aldehydes may then be reduced via sodium borohydride. The conjugate may then be purified, e.g., by ultrafiltration/diafiltration.

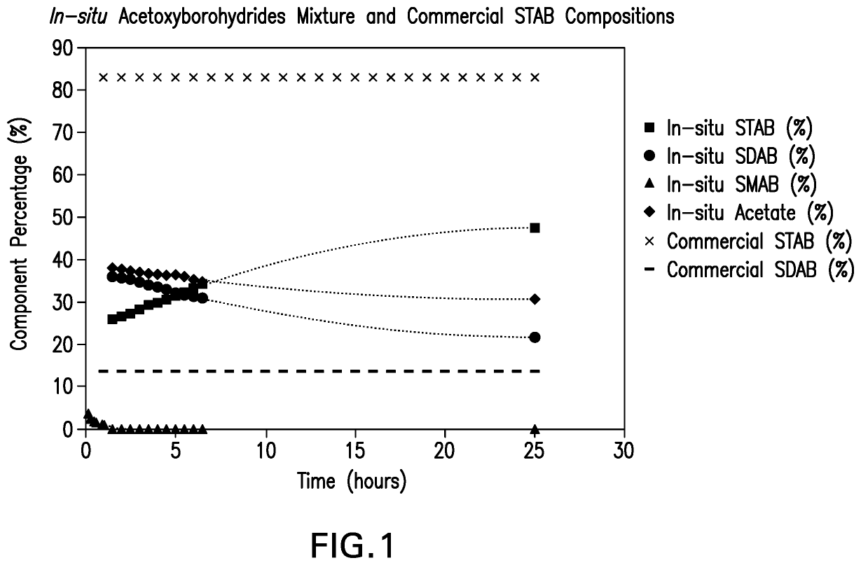

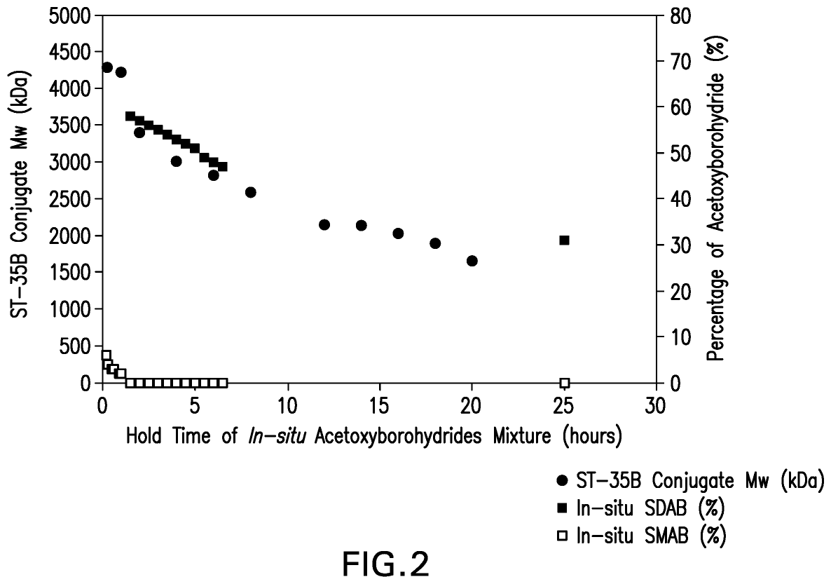

However, as noted in EP 2683408B1, the use of cyanoborohydride ions has certain disadvantages including relatively slow reaction time and the possibility of contamination of the conjugate with cyanide (Ahmed F. et al., J. Org. Chem., 1996. 61:3849-3862). Use of microwave radiation has been suggested as a solution for the slow reaction time (EP 1035137). EP 2683408B1 suggests the use of triacetoxyborohydride (BH(OA)₃) as an alternative to cyanoborohydride, since reductive amination is quicker with triacetoxyborohydride anions than the equivalent reaction using cyanoborohydride ions and toxic by-products are not produced. The suggested use of triacetoxyborohydride in the conjugation of bacterial capsular polysaccharides is based on prior reports of its use in the reductive amination of aldehydes and ketones (J. Org. Chem., 1996, 61:3849-3862) and carbohydrates (Dalpathado et al., Anal. Bioanal. Chem. (2005) 281:130-1137) The present inventors found, however, that the process of reductive amination using triacetoxyborohydride obtained from commercial sources was not very effective, and an improvement in the process was identified. This improvement comprises carrying out reductive amination using a freshly prepared acetoxyborohydride-containing reducing mixture. It was observed that reducing mixture prepared by mixing sodium borohydride and acetic acid, when fresh—within about 15 hours of preparation—contains a higher concentration of DAB (diacetoxyborohydride) than the triacetoxyborohydride reagent obtained commercially. See FIG. 1. It was observed also that with time, the concentration of DAB in the reducing mixture decreases (FIG. 1), and this decrease parallels the reduction in the size of the conjugate produced. See FIG. 2. In other words, a reducing mixture containing higher concentration of DAB leads to greater amounts of large-size conjugates. It should be noted that among MAB, DAB, and TAB, MAB has the highest reduction potential, DAB the next highest, and TAB the least reduction potential. Since the in situ prepared acetoxyborohydrides reducing mixture has relatively higher proportions of acetoxyborohydride species with higher reduction potential compared to commercial TAB, improved conjugation and conjugation attributes are observed when the in-situ prepared acetoxyborohydrides reducing mixture is used (Gordon W. Gribble. Chem. Soc. Rev., 1998, 27:395-404).

Also, it was observed that holding the reaction mixture for a period, e.g., one hour, before using it in the conjugation reaction, leads to consistency in conjugate size, and thus consistency in manufacture. Various aspects of conjugating a saccharide antigen using the freshly prepared acetoxyborohydrides reducing mixture described herein are set forth in greater detail in the numbered embodiments that follow.

Embodiment 1 provides a method for conjugating an antigen to a carrier protein, the method comprising the steps of
a) activating the antigen to form an activated antigen;
b) reacting the activated antigen with a carrier protein to obtain an intermediate, wherein the activated antigen and the carrier protein are linked by an imine group; and
c) reducing the imine group by a process comprising
   (i) mixing acetic acid with a borohydride solution to prepare a reducing mixture comprising acetoxyborohydrides, and
   (ii) treating the intermediate with the reducing mixture, wherein at least about 20% of the acetoxyborohydride in the reducing mixture is diacetoxyborohydride;
      thereby yielding a conjugated antigen, wherein the antigen is a saccharide.

Embodiment 2 provides the method of embodiment 1, wherein in step (ii), the remainder of the acetoxyborohydride in the reducing mixture is either tricetoxyborohydride or a mixture of tricetoxyborohydride and monoacetoxyborohydride.

Embodiment 3 provides the method of embodiments 1 or 2, wherein the saccharide is a bacterial capsular polysaccharide.

Embodiment 4 provides the method of any of embodiments 1-3, wherein the reducing mixture is held for a period of at least 30 minutes before treating the intermediate with the reducing mixture.

Embodiment 5 provides the method of any of embodiments 1-4, wherein the borohydride is sodium borohydride or potassium borohydride.

Embodiment 6 provides the method of any of embodiments 1-5, wherein the borohydride solution is prepared by dissolving the borohydride in dimethylsulphoxide (DMSO). Non-aqueous solvents such as acetonitrile. 1,2-dimethoxyethane, or other suitable non-aqueous solvents known to one of ordinary skill in the art may alternatively be used.

Embodiment 7 provides the method of any of embodiments 1-6, wherein step (c)(i) is carried out at a temperature of about 20° C. to about 35° C.

Embodiment 8 provides the method of embodiment 7, wherein step (c)(i) is carried out at a temperature of about 20° C. to about 25° C.

Embodiment 9 provides the method of any of embodiments 1-8, wherein unreacted carbonyl groups (e.g., residual aldehyde groups) are reduced by the reduction mixture.

Embodiment 10 provides the method of any of embodiments 1-8, further comprising reducing unreacted carbonyl groups using a borohydride, for example, sodium or potassium borohydride.

Embodiment 11 provides the method of any of embodiments 1-10, wherein the reducing mixture is held for about 1 to about 8 hours before use.

Embodiment 12 provides the method of embodiment 11, wherein the reducing mixture is held for about 2 to about 6 hours before use.

Embodiment 13 provides the method of any of embodiments 1-12, wherein the reducing mixture comprises at least about 25% diacetoxyborohydride or at least about 30% diacetoxyborohydride.

Embodiment 14 provides the method of any embodiments 1-13, wherein the bacterial capsular saccharide originates from Streptococcus pneumoniae. Haemophilus influenzae, Neisseria meningitidis, Staphylococcus aureus, Enterococcus faecium, Enterococcus faecalis, Salmonella vi, or Staphylococcus epidermidis.

Embodiment 15 provides the method of embodiment 14, wherein the bacterial capsular polysaccharide originates from Streptococcus pneumoniae (S. pneumoniae).

Embodiment 16 provides the method of embodiment 15, wherein the S. pneumoniae capsular polysaccharide is of a serotype selected from the group consisting of 1, 2, 3, 4, 5, 6A, 6B, 7F, 8, 9N, 9V, 10A, 11A, 12F, 14, 15A, 15B, deOAc-15B, 16F, 17F, 18C, 19A, 19F, 20A, 22F, 23A, 23F. 23B, 24F, 31, 24F. 31, 33F, and 35B.

Embodiment 17 provides the method of embodiment 14, wherein the bacterial capsular saccharide originates from N. meningitidis.

Embodiment 18 provides the method of embodiment 17, wherein the N. meningitidis capsular polysaccharide is of a serotype selected from the group consisting of A, B, C, W135, X, and Y.

Embodiment 19 provides the method any of embodiments 1-18, wherein the carrier protein is a protein selected from the group consisting of tetanus toxoid (TT), fragment C of tetanus toxoid, diphtheria toxoid (DT), CRM197, Pneumolysin (Ply). protein D, PhtD (Pneumococcal histidine triad protein D), PhtDE, and N19.

Embodiment 20 provides the method of embodiment 19, wherein the carrier protein is CRM197.

Embodiment 21 provides the method of embodiment 20, wherein the saccharide is conjugated to lysine residues of CRM197 to yield a molar ratio of conjugated CRM197 lysine residues to total CRM197 amine residues of between about 0.5:10 to about 5:10.

Embodiment 22 provides the method any of embodiments 1-21, wherein the conjugated antigen has a molecular weight of between about 50 kDa and about 20,000 kDa.

Embodiment 23 provides the method any embodiments 1-22, wherein the conjugated antigen comprises less than about 45% free bacterial capsular polysaccharide compared to the total amount of the bacterial capsular polysaccharide saccharide.

### Pharmaceutical Composition

In another aspect, the disclosure provides an immunogenic composition comprising a glycoconjugate of the disclosure and at least one of an adjuvant, diluent, or carrier.

In some embodiments, the disclosure provides an immunogenic composition comprising a glycoconjugate of the disclosure and at least one of an adjuvant, diluent, or carrier, wherein the glycoconjugate comprises a bacterial capsular polysaccharide covalently conjugated to a carrier protein. In some such embodiments, the capsular polysaccharide is derived from S. pneumoniae or N. meningitidis.

In some embodiments, the immunogenic composition comprises an adjuvant. In some of such embodiments, the adjuvant is an aluminum-based adjuvant selected from the group consisting of aluminum phosphate, aluminum sulfate, and aluminum hydroxide. In one embodiment, the immunogenic composition comprises the adjuvant aluminum phosphate.

In some embodiments, the immunogenic composition comprises a conjugated antigen or a mixture of conjugated antigens, one or more of which are prepared according to the method of any of the numbered embodiments 1-23 described above, mixed with a pharmaceutically acceptable excipient.

In some embodiments, the glycoconjugates or immunogenic compositions of the disclosure can be used to generate antibodies that are functional as measured by killing bacteria in an animal efficacy model or via an opsonophagocytic killing assay.

In another aspect, the disclosure provides a method of inducing an immune response in a subject, the method comprising administering to the subject an immunologically effective amount of an immunogenic composition of the disclosure as described herein. In related embodiments, the disclosure provides a method for inducing an immune response against a pathogenic bacterium in a subject, the method comprising administering to the subject an immunologically effective amount of an immunogenic composition as described herein.

In another aspect, the disclosure provides a method for preventing or ameliorating a disease or condition caused by a pathogenic bacterium in a subject, the method comprising administering to the subject an immunologically effective amount of an immunogenic composition as described herein.

In another aspect, the disclosure provides a method for reducing the severity of at least one symptom of a disease or condition caused by infection with a pathogenic bacterium in a subject, comprising administering to the subject an immunologically effective amount of an immunogenic composition as described herein.

In some embodiments, the pathogenic bacterium is S. pneumoniae or N. meningitidis. In one embodiment, the pathogenic bacterium is S. pneumoniae and the capsular polysaccharide is of a serotype selected from the group consisting of 1, 2, 3, 4, 5, 6A, 6B, 7F, 8, 9N, 9V, 10A, 1 IA, 12F, 14, 15A, 15B, DeOAc15B, 16F, 17F, 18C. 19A, 19F, 20, 22F. 23A. 23F, 23B, 24F. 31, 24F, 31, 33F and 35B. In one embodiment, the pathogenic bacterium is N. meningitidis and the capsular polysaccharide is selected from the group consisting of serotype A, B, C, W135, X and Y capsular polysaccharides of N. meningitidis.

The present invention will be more clearly understood in light of the following examples which serve to illustrate the invention without, however, limiting the contents thereof.

### EXAMPLES

### Example 1: Preparation of In-Situ Acetoxy Borohydrides Reducing Mixture

An in-situ acetoxyborohydrides reducing mixture was prepared by adding 0.4 M to 0.8 M of sodium borohydride to DMSO and mixing until the sodium borohydride was completely dissolved. Next, about 3 to 5 molar equivalents of acetic acid was added to the sodium borohydride solution to form the in-situ acetoxyborohydrides reducing mixture. The reducing mixture contains three reducing species, namely, MAB. DAB, and TAB. These species differ in their reduction potential. Further, the concentration of each species in the mixture varies with time and temperature.

The reaction to prepare the acetoxyborohydrides reducing mixture is exothermic. The rise in temperature during preparation of the reducing mixture was limited by adding acetic acid in multiple steps (between 4 to 8 additions). The preparation was carried out in a glove box to prevent incorporation of moisture. Moisture induces hydrolyzation of the reductive species and further increases the temperature of the reaction. Upon completion of the preparation, the reducing mixture was held (i.e., set aside) for about 2-8 hours before using it in a conjugation reaction. It was observed that in each preparation of the reducing mixture, by the holding the mixture for the same amount of time before use, it is possible to have approximately the same concentration of DAB in the mixture.

### Example 2: Composition of In-Situ Acetoxyborohydrides Reducing Mixture

The composition of the in-situ acetoxyborohydrides reducing mixture is time dependent. The amounts of the components. MAB, DAB, and TAB, in the mixture vary with time. These amounts can be measured by NMR. In this example, measurement of the composition of the reducing mixture was begun after the final addition of acetic acid to the borohydride solution. MAB is the most unstable species in the reducing mixture. It is consumed within the first two hours of the reaction (see FIG. 1). The concentration of DAB in the reducing mixture decreases with time, but the rate at which the decrease occurs slows down. This suggests that eventually the DAB concentration reaches a plateau. On the other hand, the concentration of TAB increases with time as MAB and DAB become converted into TAB.

### Example 3: Correlation Between Conjugate Size and the Concentration of DAB in the In-Situ Acetoxyborohydrides Reducing Mixture

Studies were carried out to examine if there is a correlation between the concentration of DAB in the in-situ acetoxyborohydrides reducing mixture and conjugate size. Conjugate size reflects the efficiency of the conjugation process. The greater the conjugate size the more efficient the process. In one study, using the bacterial capsular polysaccharide ST-35B as the antigen, it was observed that with time, as the concentration of DAB in the reducing mixture fell, the size of the ST-35B conjugate also fell (see FIG. 2). Observation was carried out over 25 hours. The fact that during this interval, the amount of TAB in the reducing mixture increased, but that of DAB fell, strongly suggests that most of the reductive amination occurring during conjugate formation is due to reduction by DAB, not TAB. In other words, DAB, rather than TAB, is the primary reducing species. Note that the level of MAB reaches zero within about 1-2 hours of the preparation of the reducing mixture.

Conjugation of several S. pneumoniae serotypes was carried out using the in-situ acetoxyborohydrides reducing mixture. These S. pneumoniae serotypes and the results of conjugation are shown in Table 1 below.

TABLE 1

| | | | Free | Residual | Lysine | | |
|---|---|---|---|---|---|---|---|
| | Mw | Mn | Protein | Aldehyde | Loss | | Free |
| Serotype | (kDa) | (kDa) | (%) | (%) | (mol/mol) | Ps:Pr | Ps % |
| ST-1 | 2432 | 2319 | <1 | 3.5 | 8.0 | 1.4 | 2.0 |
| ST-3 | 3207 | 2515 | <3 | 1.4 | 8.4 | 1.3 | N/A |
| ST-4 | 4333 | 3046 | <1 | 8.5 | 8.3 | 1.5 | 13.0 |
| ST-5 | 4805 | 3252 | <1 | 9.7 | 8.4 | 0.9 | 7.9 |
| ST-6A | 2583 | 1613 | <2 | 0.2 | 12.2 | 1.2 | 5.2 |
| ST-9N | 3553 | 2126 | 0.7 | 1.3 | 10.8 | 1.2 | 0.1 |
| ST-9V | 3674 | 1680 | <1 | 2.2 | 11.1 | 1.1 | 0.5 |
| ST-19A | 3753 | 2035 | <1 | 1.5 | 10.2 | 0.9 | 1.1 |
| ST-22F | 2639 | 2326 | <1 | 0.7 | 11.5 | 1.3 | 0.1 |
| ST-35B | 3752 | 3254 | 7.2 | 0.4 | 6.8 | 2.3 | 4.5 |

### Example 4: Comparison of In-Situ Acetoxyborohydrides Reducing Mixture with Commercial STAB

Concentrations of MAB, DAB, and TAB in the in-situ DAB reducing mixture was compared to the concentrations of these species in commercial STAB preparations. As, shown in FIG. 1, in one of the commercial TAB tested, DAB and TAB are present at about 17% and 83%, respectively. Commercial TAB preparation does not contain any MAB, which is as expected since MAB has low stability and becomes converted to TAB in about 1-2 hours.

Given that DAB is the primary reducing species (see Example 3) and given that DAB is present in the in-situ acetoxyborohydrides reducing mixture at a higher level than in the commercial STAB (at least during the 25 hours after preparation; see FIG. 1), the in-situ acetoxyborohydrides reducing mixture is a belier reducing agent than the commercial TAB. This was confirmed by a direct comparison of the conjugate size obtained by using the in-situ acetoxyborohydrides reducing mixture described herein and that obtained by using commercial STAB (see Table 2). Same molar equivalents of each of the commercial and the in-situ acetoxyborohydrides were used for the comparison. The results obtained using ST-35B, ST-9N and ST-22F as the bacterial polysaccharides are shown in Table 2 below.

TABLE 2

| | | | | Free | Residual | Lysine | |
|---|---|---|---|---|---|---|---|
| | | Mw | Mn | Protein | Aldehyde | Loss | |
| Serotype | Reducing agent | (kDa) | (kDa) | (%) | (%) | (mol/mol) | Ps:Pr |
| 35B | No reducing agent | 742 | 411 | 15% | 0.3 | 5.8 | 2.1 |
| | Sodium | 1166 | 603 | 15% | 0.3 | 5.9 | 2.1 |
| | Cyanoborohydride | | | | | | |
| | In-situ Acetoxy- | 3437 | 2004 | 4% | 0.3 | 7.5 | 2.1 |
| | borohydrides | | | | | | |
| | Mixture | | | | | | |
| | Commercial STAB | 957 | 534 | 7% | 0.2 | 6.6 | 2.5 |
| 9N | No reducing agent | 1010 | 631 | <2% | 0.9 | 13.9 | 1.3 |
| | Sodium | 1157 | 702 | <2% | 1.1 | 14.3 | 1.2 |
| | Cyanoborohydride | | | | | | |
| | In-situ Acetoxy- | 8073 | 4298 | <1% | 1.7 | 10.2 | 1.1 |
| | borohydrides | | | | | | |
| | Mixture | | | | | | |
| | Commercial STAB | 1242 | 734 | <1% | 1.2 | 13.5 | 1.2 |
| 22F | No reducing agent | 1339 | 774 | <2% | 0.8 | 10.9 | 1.3 |
| | Sodium | 1492 | 836 | <2% | 0.9 | 10.9 | 1.3 |
| | Cyanoborohydride | | | | | | |
| | In-situ Acetoxy- | 2352 | 1182 | <2% | 0.8 | 11.1 | 1.3 |
| | borohydrides | | | | | | |
| | Mixture | | | | | | |
| | Commercial STAB | 2035 | 1217 | <3% | 0.8 | 10.4 | 1.3 |

The results show that the in-situ acetoxyborohydrides reducing mixture leads to a much higher conjugate size, comparable saccharide to the carrier protein ratio (Ps:Pr), and a much lower % free polysaccharide.

### Example 5: Effect of Temperature and Hold Time of In-Situ Acetoxyborohydrides Reducing Mixture on Conjugate Size

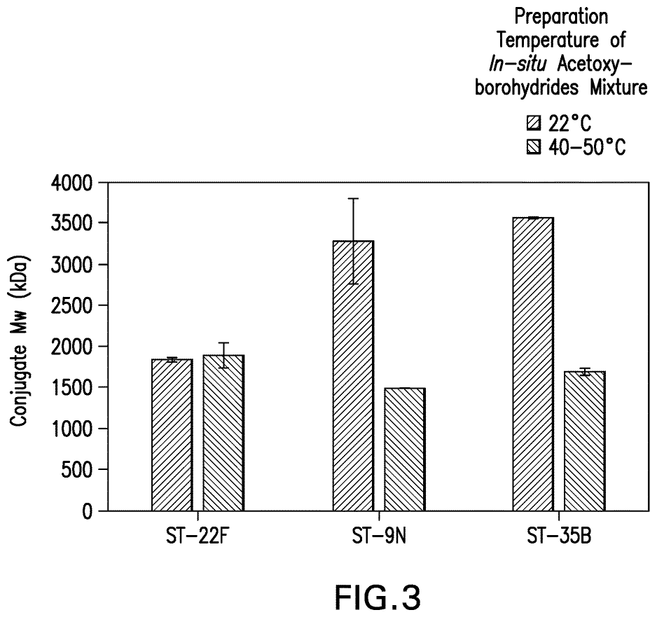

The effect of the preparation temperature of the in-situ acetoxyborohydrides mixture on conjugate size was assessed by making the reducing mixture at 22° C. or between 4° and 50° C. Three different serotypes of S. pneumoniae. ST-22F, ST-9N, and ST-35B were used in this study. The results are shown in FIG. 3. Conjugation was more effective for ST-9N and ST-35B when the reducing mixture was prepared at 22° C. instead of 40-50° C., whereas in the case of ST-22F, there was no difference.

The size of the conjugate obtained using the in-situ acetoxyborohydrides reducing mixture depends upon the reduction potential of the mixture. Among the three reducing species in the reducing mixture, SMAB has the highest reduction potential (Gribble G W, Chemical Society Reviews, 1998, volume 27, pp. 395-404) and may lead to conjugate sizes that are greater than the desired size. As such, it is desirable to hold the reducing mixture for a period of time after preparation before using it in order for the SMAB amount to be reduced or fully depleted. With time, however, the amount of SDAB in the reducing mixture decreases leading to reduced conjugate size. The latter effect is illustrated in Table 3 below using conjugation of the bacterial capsular polysaccharides 9N, 22F, and 35B.

TABLE 3

| 9N | | 22F | | 35B | |
|---|---|---|---|---|---|
| Hold Time of | | Hold Time of | | Hold Time of | |
| In-situ | | In-situ | | In-situ | |
| Acetoxy- | | Acetoxy- | | Acetoxy- | |
| borohydrides | Conjugate | borohydrides | Conjugate | borohydrides | Conjugate |
| Mixture | Size Mw | Mixture | Size Mw | Mixture | Size Mw |
| (hours) | (kDa) | (hours) | (kDa) | (hours) | (kDa) |
| 0.5 | 4666 | 0.6 | 3282 | 0.25 | 4288 |
| 1 | 4646 | 1 | 3012 | 1 | 4220 |
| 2 | 3991 | 2 | 2303 | 2 | 3401 |
| 4 | 3259 | 4 | 2626 | 4 | 3013 |

### Example 6: Results of Immunization of Mice with Pneumococcal Conjugates Prepared Using the in-Situ Acetoxyborohydrides Reducing Mixture

Young female CD1 mice (6-8 weeks old, n=10/group) were immunized intramuscularly with 0.1 ml of a 9N-CRM197, 22F-CRM197, or 35B-CRM197 vaccine on day 0 and day 14, 9N-CRM197, 22F-CRM197, or 35B-CRM197 vaccines were dosed at 0.4 μg of 9N, 22F, or 35B polysaccharide conjugated to CRM197 per immunization. Sera were collected prior to study start (pre-immune) and on day 21 (post-dose 2, PD2). Mice were observed at least daily by trained animal care staff for any signs of illness or distress. The vaccine formulations in mice were deemed to be safe and well tolerated, as no vaccine-related adverse event was noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc.

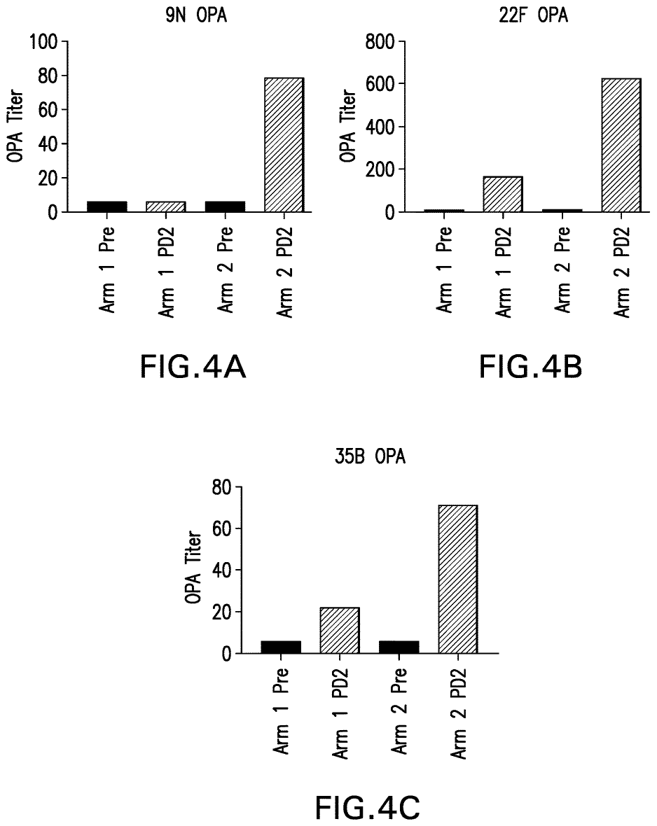

Functional antibody levels were determined using opsonophagocytic assays (OPA) based on previously described protocols at www.vaccine.uab.edu and the software Opsotiter® owned by and licensed from University of Alabama (UAB) Research Foundation, and is briefly described in the following. Mouse sera were pooled for each group and tested using OPA to determine functional antibody titers following two immunizations. ST-9N, ST-22F and ST-35B conjugations in which the in-situ acetoxyborohydride mixture described herein was used (Arm 2) exhibited higher functional antibody titers compared to the same conjugations in which the in-situ acetoxyborohydride mixture was not used (Arm 1) (see FIG. 4A, FIG. 4B, and FIG. 4C). In Arm 1, cyanoborohydride was used as the reducing agent for serotypes 9N and 22F, and for serotype 35B, no reducing agent was used.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Various structural elements of the different embodiments and various disclosed method steps may be utilized in various combinations and permutations, and all such variants are to be considered forms of the invention. Scope of the invention is thus indicated by the appended claims rather than by the foregoing description, and all changes that come within the meaning and range of equivalency of the claims are intended to be embraced therein.

What is claimed is:

1. A method for conjugating an antigen to a carrier protein, the method comprising the steps of
a) activating the antigen to form an activated antigen;
b) reacting the activated antigen with the carrier protein to obtain an intermediate, wherein the activated antigen and the carrier protein are linked by an imine group; and
c) reducing the imine group by a process comprising
(i) mixing acetic acid with a borohydride solution to prepare a reducing mixture comprising acetoxyborohydrides, and
(ii) treating the intermediate with the reducing mixture, wherein at least about 20% of the acetoxyborohydride in the reducing mixture is diacetoxyborohydride;
thereby yielding a conjugated antigen, wherein the antigen is a saccharide.

2. The method of claim 1, wherein in step (ii), the remainder in the reducing mixture is either tricetoxyborohydride or a mixture of tricetoxyborohydride and monoacetoxyborohydride.

3. The method of claim 1, wherein the saccharide is a bacterial capsular polysaccharide.

4. The method of claim 1, wherein the reducing mixture is held for a period of at least 30 minutes before treating the intermediate with the reducing mixture.

5. The method of claim 1, wherein the borohydride is sodium borohydride or potassium borohydride.

6. The method of claim 1, wherein the borohydride solution is prepared by dissolving the borohydride in dimethylsulphoxide (DMSO).

7. The method of claim 1, wherein step (c)(i) is carried out at a temperature of about 20° C. to about 35° C.

8. The method of claim 7, wherein step (c)(i) is carried out at a temperature of about 20° C. to about 25° C.

9. The method of claim 1, wherein unreacted carbonyl groups are reduced by the reducing mixture.

10. The method of claim 1, further comprising reducing unreacted carbonyl groups using a borohydride.

11. The method of claim 1, wherein the reducing mixture is held for about 1 to about 8 hours before use.

12. The method of claim 11, wherein the reducing mixture is held for about 2 to about 6 hours before use.

13. The method of claim 1, wherein the reducing mixture comprises at least about 25% diacetoxyborohydride.

14. The method of claim 1, wherein the bacterial capsular saccharide originates from Streptococcus pneumoniae, Haemophilus influenzae, Neisseria meningitidis, Staphylococcus aureus, Enterococcus faecium, Enterococcus faecalis, Salmonella vi, or Staphylococcus epidermidis.

15. The method of claim 14, wherein the bacterial capsular polysaccharide originates from Streptococcus pneumoniae (S. pneumoniae).

16. The method of claim 15, wherein the S. pneumoniae capsular polysaccharide is of a serotype selected from the group consisting of 1, 2, 3, 4, 5, 6A, 6B, 7F, 8, 9N, 9V, 10A, 11A, 12F, 14, 15A, 15B, deOAc-15B, 16F, 17F, 18C, 19A, 19F, 20, 20A, 22F, 23A, 23F, 23B, 24F, 31, 33F, and 35B.

17. The method of claim 14, wherein the bacterial capsular saccharide originates from Neisseria meningitidis (N. meningitidis).

18. The method of claim 17, wherein the N. meningitidis capsular polysaccharide is of a serotype selected from the group consisting of A, B, C, W135, X, and Y.

19. The method of claim 1, wherein the carrier protein is a protein selected from the group consisting of tetanus toxoid (TT), fragment C of tetanus toxoid, diphtheria toxoid (DT), CRM197, Pneumolysin (Ply), protein D, PhtD (Pneumococcal histidine triad protein D), PhtDE, and N19.

20. The method of claim 19, wherein the carrier protein is CRM197.

21. The method of claim 20, wherein the saccharide is conjugated to lysine residues of CRM197 to yield a molar ratio of conjugated CRM197 lysine residues to total CRM197 amine residues of between about 0.5:10 to about 5:10.

22. The method of claim 1, wherein the conjugated antigen has a molecular weight of between about 50 kDa and about 20,000 kDa.

23. The method of claim 1, wherein the conjugated antigen comprises less than about 45% free bacterial capsular polysaccharide compared to the total amount of the bacterial capsular polysaccharide saccharide.
